(19) United States
(12) Patent Application Publication
(10) Pub. No.: US 2017/0020909 A1
(43) Pub. Date: Jan. 26, 2017

(54) METHODS OF TREATING GLAUCOMA USING AMP-ACTIVATED PROTEIN KINASE (AMPK) ACTIVATORS

(71) Applicant: Massachusetts Eye and Ear Infirmary, Boston, MA (US)

(72) Inventors: Douglas J. Rhee, Solon, OH (US); Guadalupe Villarreal, Jr., Baltimore, MD (US); Ayan Chatterjee, Durham, NC (US); Dong-Jin Oh, Cleveland Heights, OH (US); Min Kang, Solon, OH (US)

(21) Appl. No.: 15/125,049

(22) PCT Filed: Mar. 10, 2015
(86) PCT No.: PCT/US2015/019606

§ 371 Date: Sep. 9, 2016

Related U.S. Application Data
(60) Provisional application No. 61/951,273, filed on Mar. 11, 2014.

(51) Int. Cl. A61K 31/7056; A61K 9/00

(52) U.S. Cl. CPC A61K 31/7056; A61K 9/0048

(57) ABSTRACT

Methods of reducing intraocular pressure in a mammal using AMPK activators, e.g., for treating glaucoma.

### CLAIM OF PRIORITY

This application claims the benefit of U.S. Provisional Patent Application Ser. No. 61/951,273, filed on Mar. 11, 2014, the entire contents of which are hereby incorporated by reference.

### FEDERALLY SPONSORED RESEARCH OR DEVELOPMENT

This invention was made with Government support under Grant Nos. EY 019654-01 and EY 014104 awarded by the National Eye Institute of the National Institutes of Health. The Government has certain rights in the invention.

### TECHNICAL FIELD

Described herein are methods of reducing intraocular pressure in a mammal using AMPK activators, e.g., for treating glaucoma.

### BACKGROUND

Glaucoma is a leading cause of irreversible blindness.¹ Elevated intraocular pressure (IOP) in eyes with primary open-angle glaucoma (POAG) is caused by poor aqueous humor drainage and can lead to visual field loss due to progressive optic nerve damage.² The only rigorously proven treatment for POAG is to lower IOP.^{3,4} Thus far, single gene mutations account for less than 10% of POAG cases, with the other 90% likely having polygenic origins.⁵

### SUMMARY

Complex regulatory mechanisms govern extracellular matrix (ECM) homeostasis, cellular tone, and aqueous outflow in the trabecular meshwork (TM). The present data identifies AMP-activated protein kinase (AMPK) as a regulatory element for intraocular pressure (IOP) and possible novel therapeutic target for glaucoma. A variety of pharmacologic activators of AMPK exist.

The present invention is based, at least in part, on the discovery that AMPK signaling has functional relevance to IOP homeostasis, and AMPK activators are expected to have therapeutic efficacy in human disorders of IOP homeostasis, e.g., glaucoma or a disorder listed in Table 1.

Thus, in a first aspect, the invention provides methods for reducing intraocular pressure (IOP) in a mammal. The methods include identifying a mammal in need of reduced IOP; and administering to the mammal an effective amount of an amp-activated protein kinase (AMPK) activator sufficient to reduce IOP in the mammal.

In another aspect, the invention provides methods for treating glaucoma in a mammal. The methods include identifying a mammal who has glaucoma; and administering to the mammal a therapeutically effective amount of an amp-activated protein kinase (AMPK) activator.

Also provided herein are an AMP-activated protein kinase (AMPK) activator for use in the reduction of IOP in a mammal, and the use of an amp-activated protein kinase (AMPK) activator in the manufacture of a medicament to reduce IOP in a mammal.

In some embodiments, the mammal has ocular hypertension, a primary or secondary form of acute or chronic open-angle glaucoma, a primary or secondary acute or chronic angle-closure glaucoma, and/or a congenital or developmental glaucoma.

In another aspect, the invention provides a pharmaceutical composition comprising an AMPK activator formulated for ocular administration, e.g., formulated for topical ocular administration. In some embodiments, the composition is formulated as eye drops, topical eye cream, or topical eye lotion, e.g., single use ampules, which optionally lack a preservative.

In some embodiments, the AMPK activator formulation comprises microcapsules, microemulsions, or nanoparticles.

In a further aspect, the invention provides containers for drop-wise dispensation of a pharmaceutical composition into the eye of a subject, the containers having disposed therein an amount of an AMPK activator. In some embodiments, the containers are single use ampules, which optionally lack a preservative.

In some embodiments, the AMPK activator is an activator described herein, e.g., selected from the group consisting of 5-Aminoimidazole-4-carboxamide riboside (AICA riboside or AICAR); AICA ribotide (ZMP); guanidine; galegine; metformin (dimethylbiguanide); phemformin (phenethylbiguanide); antifolate drugs that inhibit AICAR transformylase (e.g., methotrexate, pemetrexed); thiazolidinediones (e.g., rosiglitazone, pioglitazone, or troglitazone); 2-Deoxyglucose (2DG); phenobarbital; A-769662; PT1; salicylate; C24; A-769662 (4-hydroxy-3-[4-(2-hydroxyphenyl)phenyl]-6-oxo-7H-thieno[2,3-b]pyridine-5-carbonitrile); D942 (5-[3-[4-[2-(4-fluorophenyl)ethoxy]phenyl]propyl]furan-2-carboxylic acid); and ZLN024.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D. At 6-7 weeks of age, AMPKα2-null mice exhibit increased IOP compared to their WT counterparts, with no significant difference in central corneal thickness (CCT) or gross architecture of the iridocorneal angle. (A) IOP was obtained under sedation by TonoLab in WT (n=35) and AMPKα2-null mice (n=44). AMPKα2-null mice had on average 6.0% higher IOP than WT mice (*p=0.0265 by student t-test), 18.3±0.3 versus 17.2±0.4 mmHg (mean±SEM). (B) CCT measurements were obtained under sedation by optical coherence tomography in WT (n=35) versus AMPKα2-null mice (n=44). Data expressed as mean±SEM (p=0.6877 by student t-test; NS=not significant). Representative light microscopic images of iridocorneal angles in (C) AMPKα2-WT and (D) AMPKα2-null mice appear grossly indistinguishable with similar Schlemm's canals, trabecular beams and cellularity, uveoscleral outflow pathway, and ciliary body location. AC, anterior chamber; TM, trabecular meshwork; SC, Schlemm's canal; CP; ciliary processes. Scale bar, 50 μm. All tissues stained with toluidine blue.

FIGS. 2A-B. At 7 weeks of age, AMPKα2-null mice exhibit decreased aqueous humor clearance. (A) Representative series of green channel images captured at 10-minute intervals after corneal permeabilization with 0.02% BAC followed by topical application of 0.02% fluorescein and saline wash (green in original). (B) Aqueous fluorescein levels relative to values at t=0 for WT and AMPKα2-null mice (n=7 and n=5, respectively). Least-squares fit for exponential decay yielded (% intensity)=100e-0.1112*time, and (% intensity)=100e-0.0854*time for WT and AMPKα2-null, respectively. *Significant differences were observed in relative intensities between WT and AMPKα2-null mice at 10 minutes, 20 minutes, 30 minutes, and 40 minutes (p=0.044, 0.037, 0.049, 0.049, respectively). Data expressed as mean relative intensity (%)±SEM.

FIGS. 3A-B. AMPKα1 and AMPKα2 are expressed in human TM. (A) Representative immunoblots show detection of p-AMPKα, AMPKα1, and AMPKα2 in cell lysates of primary cultured human TM cells (n=4), each at approximately 62 kDa. (B) Representative immunofluorescent staining of AMPKα1 and AMPKα2 in sections of adult human cadaveric donor eyes (n=4). Nuclei were stained with DAPI. AC, anterior chamber; TM, trabecular meshwork; SC, Schlemm's canal. Scale bar, 50 μm.

FIGS. 4A-B. AICAR treatment leads to phosphorylation and activation of AMPKα. Primary cultured human TM cells were lysed at the specified time intervals after treatment with 0.5 mM AICAR. (A) Representative immunoblots of cell lysates showing detection of p-AMPKα (Thr172), total AMPKα, p-ACC, and total ACC with β-actin loading control. AMPKα antibodies detect both α1 and α2 isoforms. (B) Integrated band intensities calculated from above immunoblots. Data expressed as mean phospho/total ratios (normalized to zero time point)±SEM (*p<0.05 vs. zero time point by student t-test; n=4).

FIGS. 5A-E. AICAR suppresses ECM proteins in primary human TM cells under basal and TGF-β2 stimulatory conditions. (A) Representative immunoblots of ECM proteins from conditioned media (CM) of human TM cells treated for 24 hours with PBS vehicle or 0.5 mM AICAR and (B) integrated band intensities calculated from those immunoblots. (C) Representative immunoblots of ECM proteins from CM of human TM cells under stimulation with 2.5 ng/mL TGF-β2. Cells were pre-incubated for 1 hour with PBS or 0.5 mM AICAR prior to full 24-hour treatment. (D) Mean integrated band intensities. Data in panels B and D are expressed as mean±SEM (*p<0.05 vs. PBS vehicle by student t-test; n=5-7). (E) Representative 10% acrylamide gels stained with Coomassie Brilliant Blue as a loading control.

FIG. 6. Under basal and TGF-β2 stimulatory conditions, AICAR treatment leads to decreased F-actin cytoskeletal staining, and fewer actin stress fibers. Human TM cells were plated on 8-well slides, treated as in FIG. 3, and then stained with phalloidin (F-actin) antibody. Nuclei were stained with DAPI. Representative immunofluorescent images shown above (n=3). Scale bar, 50 μm.

FIGS. 7A-B. AICAR treatment leads to phosphorylation of RhoA. Human TM cells were lysed at the specified time intervals after treatment with 0.5 mM AICAR. (A) Representative immunoblots of cell lysates showing detection of p-RhoA (Ser188) and total RhoA, with β-actin loading control. (B) Integrated band intensities calculated from above immunoblots. Data expressed as mean phospho-total ratios (normalized to zero time point)±SEM (*p<0.05 vs. zero time point by student t-test; n=3).

FIGS. 8A-B. TGF-β2 treatment leads to transient dephosphorylation of AMPKα in human TM cells. TM cells were lysed at the specified time intervals after treatment with 2.5 ng/mL TGF-β2. (A) Representative immunoblots of cell lysates showing detection of p-AMPKα (Thr172) and total AMPKα, with β-actin loading control. Antibodies detect both α1 and α2 isoforms. (B) Mean integrated band intensities calculated from above immunoblots. Data expressed as mean phospho-total ratios (normalized to zero time point)±SEM (*p<0.05 vs. zero time point by student t-test; n=4). Analysis reveals that the p-AMPKα/AMPKα ratio is significantly decreased only at the t=15 minutes time point (p=0.0067).

FIGS. 9A-F. Adenoviral transfer of a dominant negative form of the AMPKα subunit (ad.DN.AMPKα) increases matricellular and ECM expression, decreases the phospho-total RhoA ratio (Ser188), and increases F-actin cytoskeletal staining and disarray. (A) Representative immunoblots of ECM proteins from CM of human TM cells treated for 66 hours with null adenoviral vector (ad.null) versus ad.DN.AMPKα at 25 MOI. (B) Mean±SEM integrated band intensities calculated from those immunoblots (*p<0.05 by student t-test; n=4-6). (C) Representative 10% acrylamide gels stained with Coomassie Brilliant Blue as a loading control. (D) Representative immunoblots of lysates from cells treated as described in A; probed for p-RhoA, RhoA, Myc-Tag for confirmation of adenoviral expresion, and β-actin loading control. (E) Mean integrated band intensities, showing a 27% decrease in the phospho-total RhoA ratio (p=0.0053; n=7). (F) Representative images of primary cultured human TM cells plated on 8-well slides and treated as in panel A, and then stained for F-actin. Nuclei were stained with DAPI. Representative immunofluorescent images shown above (n=3). Scale bar, 50 μm.

FIG. 10. Theoretical model for the role of AMPK signaling in the regulation of ECM homeostasis and cellular tone in TM. Treatment with pharmacologic activators of AMPK results in phosphorylation of the a subunit at Thr172. Activation of AMPK leads to phosphorylation of RhoA at Ser188, as demonstrated previously in nonocular tissue (Gayard et al., Arterioscler. Thromb. Vasc. Biol. 2011; 31:2634-2642). Phosphorylation of RhoA at Ser188 results in decreased interaction with ROCK and subsequent decrease in ECM deposition. In addition, cells adopt a more unidirectional cytoskeletal arrangements with less prominent F-actin staining. With decreased ECM deposition in the TM and weaker intracellular actin stress fibers, aqueous humor outflow facility is enhanced and IOP is consequently reduced.

FIG. 11. Treatment of constant-flow-perfused ex vivo human anterior segments with 2.5 μL of 200 mM AICAR (in 1 mL of ex vivo media) resulted in a mean decrease in IOP of 18.54±1.78% by day 7, compared with paired opposite eye controls. Representative plot (n=3). Data expressed as mean percentage change in IOP±SEM (p<0.05 for paired t-tests).

### DETAILED DESCRIPTION

In humans, approximately 80-90% of aqueous outflow occurs through the TM (conventional pathway) with the remaining 10-20% exiting through the ciliary body face (alternative pathway).⁶ In mice a greater proportion of outflow occurs via the alternative pathway.^{7, 8} The juxtacanalicular (JCT) region of the TM, an amorphous layer composed of endothelial cells and extracellular matrix (ECM), is thought to be where the regulation of aqueous outflow takes place.⁹ Under conditions of elevated IOP, the JCT has the highest outflow resistance.¹⁰ The ECM within the JCT is constantly being remodeled.¹¹

The regulation of IOP in the JCT region is a complex system. Some processes, such as the regulation of ECM homeostasis, have been shown to influence IOP.¹²⁻¹⁷ Modifications in the actin cytoskeleton and cellular tone of the JCT TM and inner wall of Schlemm's canal cells have also been shown to affect IOP¹⁸ by contributing to changes in aqueous outflow facility.^{19, 20} In non-glaucomatous eyes, increasing ECM production or slowing its turnover alters IOP, and alterations of the JCT ECM constitute primary pathophysiologic events.^{14, 15, 21, 22}

Matricellular proteins are nonstructural secreted glycoproteins that facilitate cellular control over the surrounding ECM. SPARC (secreted protein acidic and rich in cysteine)—the prototypical matricellular protein—is widely expressed in human ocular tissues, including TM endothelial cells.^{23, 24} Overexpression of SPARC by TM cells increases IOP in perfused cadaveric human anterior segments derived from nonglaucomatous eyes.²⁵ This elevation of IOP coincides with an increase of certain ECM proteins within the JCT. Conversely, SPARC-null mice demonstrate 15-20% lower IOP than their wild-type (WT) counterparts as a result of increased aqueous clearance²⁶ due, in part, to greater areas of high flow TM.²⁷ Thrombospondin-1 (TSP-1), like SPARC, is also a matricellular protein expressed in the TM.^{28,29} TSP-1 null mice have a 10% lower IOP than their WT counterparts.³⁰ Elucidation of upstream regulators of proteins such as SPARC and TSP-1 may lead to new therapeutic targets.

AMP-activated kinase (AMPK) is a highly conserved serine/threonine protein kinase that regulates cellular metabolism, proliferation, and aging processes.³¹⁻³³ It exists throughout the eukaryotic domain as heterotrimeric complexes uniting a catalytic α subunit with regulatory β and γ subunits.³⁴ Within the mammalian kingdom, each subunit has multiple isoforms—α1 and α2; =1 and β2; γ1, γ2, and γ3; in humans, each encoded at a distinct genetic locus within the genome—yielding a total of twelve possible heterotrimeric combinations that appear to be distributed throughout the body in a tissue-specific manner.³⁵ Interestingly, elderly men have been shown to have reduced expression of the α2 isoform in skeletal muscle compared to younger men.³⁶ Additionally, both endurance training³⁷ and enhancement of thyroid function³⁸ generally appear to increase α2 activity in a variety of skeletal muscle types. Aging in general has been demonstrated to impair insulin-stimulated glucose uptake by suppressing AMPKα activity.³⁹ The role of AMPK in diabetes, atherosclerosis, and cancer progression has made it an attractive pharmaceutical target.^{31, 34} Although AMPK signaling has been studied in ocular diseases such as diabetic retinopathy⁴⁰⁻⁴², its potential role in ECM homeostasis in the TM, IOP regulation, and glaucoma progression is unknown.

Transforming growth factor-β2 (TGF-β2) is greatly increased in the aqueous humor of patients with POAG compared with age-matched controls^{43, 44}, and several studies suggest that TGF-β2-mediated fibrosis contributes to POAG pathogenesis.⁴⁵⁻⁴⁷ We have shown that TGF-β2 upregulates SPARC expression in human TM cells.⁴⁸ AMPK regulates matrix remodeling following injury to various non-ocular tissues^{31, 49-51}, and its signaling pathways interact with TGF-β2 during inflammation⁵², angiogenesis⁵³, and fibrosis⁴⁹. Pharmacologic activation of AMPK has been shown to suppress TGF-β2-induced fibrosis in liver.⁴⁹ We hypothesized that AMPK has functional relevance to IOP and that at least part of its mechanism involves altering SPARC, TSP-1, and other select ECM proteins. We evaluated IOP and aqueous humor clearance in mice harboring single gene deletions in the catalytic α2 subunit of AMPK and examined the effects of AMPK modulation on matricellular and ECM protein levels under basal and TGF-β2 stimulatory conditions in TM endothelial cells.

### The Role of AMPK in Regulating Intraocular Pressure, Extracellular Matrix, and Cytoskeleton in Trabecular Meshwork

As shown herein, AMPKα2-null mice have higher IOPs than their WT counterparts, which does not appear to be an artifact of CCT. The absence of gross structural differences in the iridocorneal angles implicates cellular or biochemical processes. IOP elevation may be the result of two possible mechanisms, decreased aqueous outflow facility or increased aqueous production. The decreased aqueous humor clearance exhibited by AMPKα2-null mice suggests that reduced outflow facility is the underlying mechanism behind the observed IOP elevation. Although decreased fluorescein disappearance could be the result of decreased aqueous production, in the setting of an elevated IOP, decreased outflow has to be part of the mechanism.

A greater proportion of outflow occurs though the pressure-independent alternative pathway in mice than in humans, but this appears to vary across strains, ranging from 20.5% in BALB/cJ mice⁷⁰ to 82% in NIH Swiss White mice.⁸ The mice used in this study were derived from C57Bl/6 mice, which have demonstrated 66% outflow through the alternative pathway.⁷ The observed variability in alternative outflow may be due to strain-specific properties, such as the degree of scleral permeability, or simply due to differences in the enucleation methodologies employed by the laboratories.⁷ Although in humans, a greater proportion of outflow is pressure-dependent, studies in younger humans (less than 30 years of age) indicate that the majority of outflow is through the alternative pathway, so the present methods are expected to be applicable to humans as well.

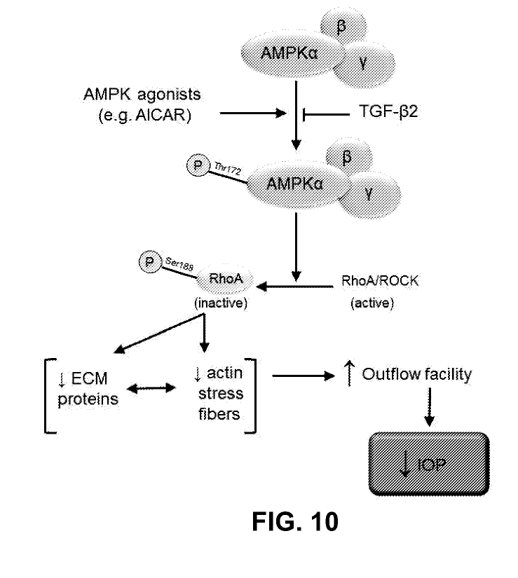

The extent to which AMPK signaling regulates ECM homeostasis and cellular tone in TM cells may explain its apparent role in aqueous clearance (FIG. 10). We demonstrated that AMPKα1 and AMPKα2 are present throughout the TM and that activation of AMPK signaling decreases certain ECM components, while resulting in narrower cells with decreased F-actin staining RhoA is a protein downstream of AMPK that unifies our findings. RhoA harbors an optimal AMPK recognition motif, and one recent investigation using controlled in vitro kinase assays provides strong evidence that AMPK directly phosphorylates RhoA in vascular smooth muscle cells (Gayard et al., Arterioscler. Thromb. Vasc. Biol. 2011; 31:2634-2642). Furthermore, although Gayard et al. describe a potential role for AMPKα1 in the phosphorylation of RhoA in mice, the relative contributions of α1 and α2 isoforms has yet to be fully explored.

In addition to altering cellular tone, RhoA induces ECM deposition in TM, thereby increasing resistance to aqueous humor outflow.^{19,20} In the prevailing model of RhoA protein activation, there is a dynamic cycle between active GTP-bound and inactive GDP-bound RhoA, and a variety of signal intermediaries favoring GTP-RhoA, which translocates to the cell membrane where it interacts with ROCK to affect ECM deposition.⁷¹ As described herein, activation of AMPK increases the phospho-total RhoA ratio (Ser188), most likely uncoupling the RhoA/ROCK pathway that normally mediates actin stress fiber formation and ECM deposition in the TM. These cytoskeletal changes are the converse of what has been reported in cells infected with adenovirus expressing constitutively active RhoA, namely more rounded morphology with increased F-actin staining.¹⁹ Furthermore, adenoviral transfer of dominant negative AMPKα resulted in cytoskeletal changes similar to those induced by RhoA overexpression. Taken together, these data suggest that AMPK—through its effects on RhoA—plays a role in both (1) ECM homeostasis and (2) cellular tone within the TM.

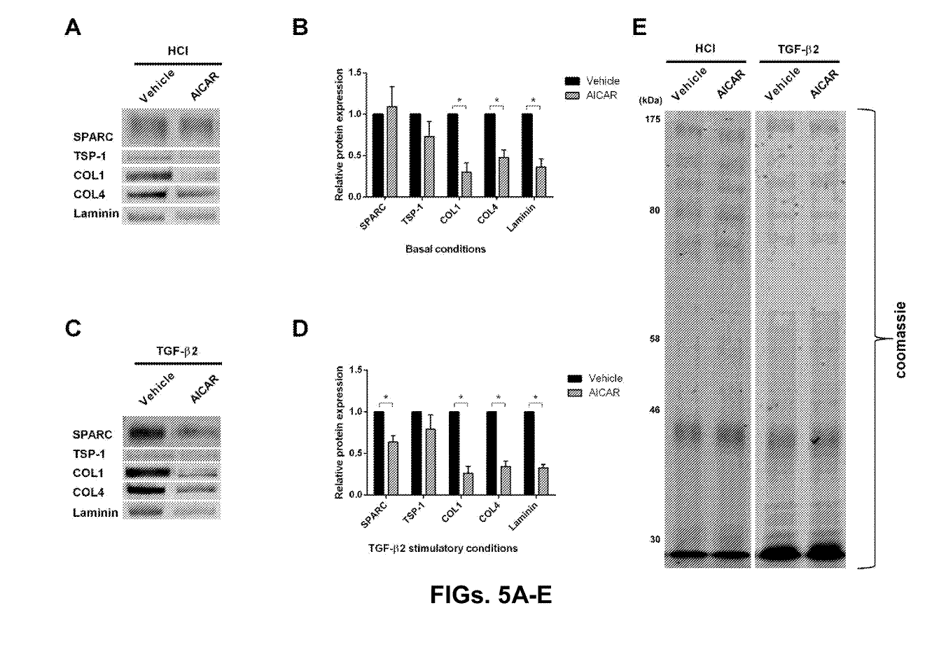

The 24-hour time frame of the results reported in FIG. 5 is more consistent with an AMPK-mediated alteration in the rate of ECM protein turnover than a decrease in the production of ECM components. Indeed, one recent investigation revealed that none of the ECM components whose protein levels were increased within 24 hours of adenoviral SPARC overexpression showed any significant, concurrent elevation in corresponding mRNA levels.²⁵ This would suggest that in the short term SPARC may be acting posttranslationally, perhaps as a chaperone molecule that stabilizes ECM components, in order to increase the efficiency of matrix deposition.⁷²⁻⁷⁵ Similarly, in the current study, it appears that the 24-hour time frame is most likely indicative of a predominantly posttranslational AMPK- and RhoA-mediated chain of intracellular and extracellular events rather than simply an increase in the transcription of ECM components.

AMPK in Human Disease

The catalytic α subunit of AMPK is expressed in human TM. It is intriguing that the genes encoding several AMPK subunits lie in close proximity to, or even within, loci that have been associated with diseases such as POAG, juvenile open-angle glaucoma, familial high myopia, pigment dispersion syndrome, pigmentary glaucoma, and pseudoexfoliation syndrome (Table 1).⁷⁸⁻⁸⁵ Thus AMPK activators may be useful for treating these conditions as well.

TABLE 1
AMPK and potential glaucoma clinical correlations from genetic studies

| Subunit | Locus | MIM | Potential disease associations | References |
|---|---|---|---|---|
| α1 | 5p12 | 602739 | N/A* | |
| α2 | 1p31 | 600497 | N/A* | |
| β1 | 12q24.1-q24.3 | 602740 | Familial high myopia | (Wojciechowski et al., 2009) |
| β2 | 1q21.1 | 602741 | POAG; JOAG; | (David et al., 1980; |
| | | | Familial high myopia | Sheffield et al., 1993; |
| | | | | Wiggs et al., 1994; |
| | | | | Wiggs et al., 1995) |
| γ1 | 12q12-q14 | 602742 | N/A* | |
| γ2 | 7q36.1 | 602743 | PDS; Pigmentary glaucoma; | (Andersen et al., 1997; |
| | | | Familial high myopia | Naiglin et al., 2002) |
| γ3 | 2q35 | 604976 | PEX | (Sotirova et al., 1999) |

MIM = Molecular Interactions Map;
POAG = Primary open-angle glaucoma;
JOAG = Juvenile open-angle glaucoma;
PDS = Pigment dispersion syndrome;
PEX = pseudoexfoliation syndrome
*To date, no known disease associations at these loci

Methods of Treatment

As described herein, AMPK signaling has functional relevance to IOP homeostasis, and AMPK activators are expected to have therapeutic efficacy in human disorders of IOP homeostasis, e.g., glaucoma or a disorder listed in Table 1. Thus, the methods described herein include methods for the treatment of disorders associated with excessive IOP. As used herein, “excessive IOP” means an intraocular pressure of greater than 21 mmHg measured in one or both eyes, e.g., measured using a tonometer, air-puff test, Goldmann tonometry, or other method, or determined to be excessive beyond the therapeutic target e.g., low tension glaucoma. In some embodiments, the disorder is glaucoma. Generally, the methods include administering a therapeutically effective amount of an AMPK activator as described herein, to a subject who is in need of, or who has been determined to be in need of, such treatment. In some embodiments, the subject does not have an inflammatory eye disease, e.g., uveitis, and/or does not have an ocular neovascularization disease or vascular leakage disease.

As used in this context, to “treat” means to ameliorate at least one symptom of the disorder associated with excessive IOP. Often, excessive IOP results in eye pain, headache, blurred vision, or the appearance of halos around lights; thus, a treatment can result in a reduction in any of those symptoms and a return or approach to normal IOP. Administration of a therapeutically effective amount of a compound described herein for the treatment of a condition associated with excessive IOP will result in decreased IOP.

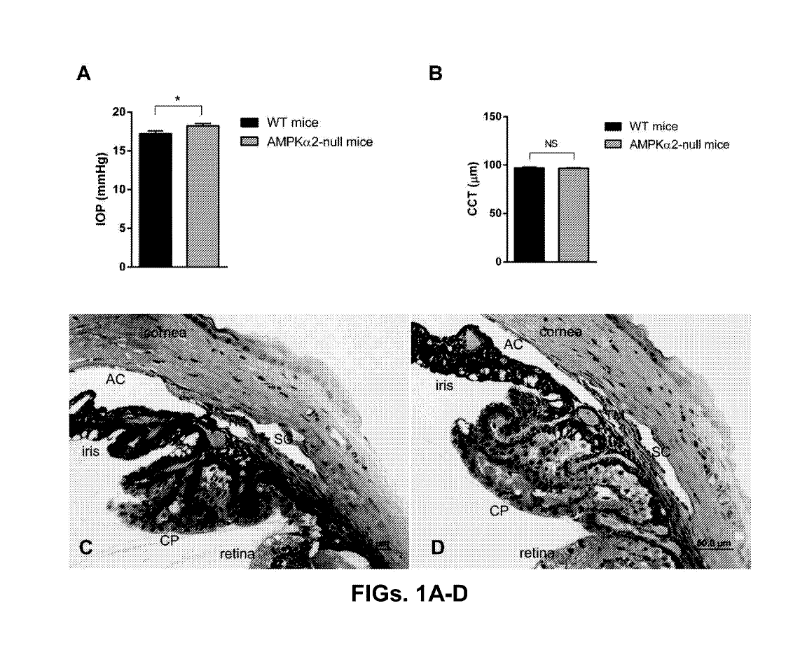

Known AMPK activators include drugs such as 5-Aminoimidazole-4-carboxamide riboside (AICA riboside or AICAR); AICA ribotide (ZMP); guanidine; galegine; metformin (dimethylbiguanide); phemformin (phenethylbiguanide); antifolate drugs that inhibit AICAR transformylase (e.g., methotrexate, pemetrexed); thiazolidinediones (e.g., rosiglitazone, pioglitazone, or troglitazone); 2-Deoxyglucose (2DG); phenobarbital; A-769662; PT1; and salicylate. See, e.g., Hardie et al. (2012) Chem. Biol. 19:1222-1236; Hawley et al. (2012) Science 336:918-922. A number of other small molecule inhibitors of AMPK are known in the art, including C24 (Li et al., Toxicol Appl Pharmacol. 2013 Dec. 1; 273(2):325-34); A-769662 (4-hydroxy-3-[4-(2-hydroxyphenyl)phenyl]-6-oxo-7H-thieno[2,3-b]pyridine-5-carbonitrile; Cool et al., Cell Metab. 2006 June; 3(6):403-16); D942 (5-[3-[4-[2-(4-fluorophenyl)ethoxy]phenyl]propyl]furan-2-carboxylic acid); ZLN024 (see FIG. 1A of Zhang et al., PLoS ONE 8(8): e72092 (2013)). In addition, AMPK activators are described in the following: U.S. Pat. No. 8,604,202B2 (Merck); U.S. Pat. No. 8,592,594B2 (Roche); U.S. Pat. No. 8,586,747B2 (Roche); U.S. Pat. No. 8,563,746B2 (Merck); U.S. Pat. No. 8,546,427B2 (Roche); U.S. Pat. No. 8,563,729B2 (Merck); U.S. Pat. No. 8,394,969B2 (Merck); U.S. Pat. No. 8,329,914B2 (Merck); U.S. Pat. No. 8,329,738B2 (Merck); US20120172333A1 (GSK); US20110060001A1 (Merck); US20090105293A1 (Merck); EP2519527B1 (Poxel); and WO2010073011A2 (Betagenon). See also WO2013003467.

In some embodiments, the AMPK activator is administered systemically, e.g., orally; in preferred embodiments, the AMPK activator is administered to the eye, e.g., via topical (eye drops, lotions, or ointments) administration, or by injection, e.g., periocular or intravitreal injection; see, e.g., Gaudana et al., AAPS J. 12(3):348-360 (2010); Fischer et al., Eur J Ophthalmol. 21 Suppl 6:S20-6 (2011). In some embodiments, the AMPK activator is administered using a device, e.g., as described in WO2004073551.

The methods described herein include the manufacture and use of pharmaceutical compositions, which include compounds identified by a method described herein as active ingredients. Also included are the pharmaceutical compositions themselves.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include systemic (e.g., parenteral and oral) and local (ocular) administration. Thus also within the scope of the present disclosure are compositions comprising the AMPK activators described herein in a formulation for administration for the eye, e.g., in eye drops, lotions, creams, e.g., comprising microcapsules, microemulsions, nanoparticles, etc. Methods of formulating suitable pharmaceutical compositions for ocular delivery are known in the art, see, e.g., Losa et al., Pharmaceutical Research 10:1 (80-87 (1993); Gasco et al., J. Pharma Biomed Anal., 7(4):433-439 (1989); Fischer et al., Eur J Ophthalmol. 21 Suppl 6:S20-6 (2011); and Tangri and Khurana, Intl J Res Pharma Biomed Sci., 2(4):1541-1442 (2011).

General methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and in some cases should be preserved against the contaminating action of microorganisms such as bacteria and fungi (the exception being non-preserved dosage forms, e.g., single dose amplules of topical drops). The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and/or sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can be included in a container, pack, or dispenser (e.g., eye drop bottle) together with instructions for administration. In some embodiments, the compositions are provided lyophilized or dry, and the kit includes saline for making a solution comprising the AMPK activator.

### Examples

The invention is further described in the following examples, which do not limit the scope of the invention described in the claims.

Materials and Methods

The following materials and methods were used in the Examples 1-6 set forth below.

Animal Care and Husbandry

All experiments were in compliance with the ARVO Statement for the Use of Animals in Ophthalmic and Vision Research and received approval from the Massachusetts Eye and Ear Infirmary animal care and use committee. AMPKα2-null mice, generously provided by Viollet and colleagues, were developed and described elsewhere.⁵⁴ Briefly, a targeting construct corresponding to the AMPKα2 catalytic domain (amino acids 189-260) was electroporated into 129/Sy MPI-I embryonic stem cells and the resultant polymerase chain reaction (PCR)-confirmed clones were injected into C57Bl/6 blastocysts. Germline-transmitting chimeric animals were mated with C57Bl/6 mice to produce heterozygous offspring, which were then crossed to produce control and mutant mice. All mice for these experiments were bred at our facility, fed ad libitum, and housed at 21° C. in clear plastic rodent cages under 12-hour light/12-hour dark cycles (on 07:00, off 19:00). Wild-type (WT) and null colonies were maintained by breeding heterozygotes with subsequent genotyping of all progeny to prevent species drift. Confirmation of homozygosity was performed as previously described²⁶, using the following PCR primer sequences: AMPKα2-WT [5′-GCTTAGCACGTTACCCTGGATGG-3′] (forward; SEQ ID NO:1) and [5′-GTTATCAGCCCAACTAATTACAC-3′] (reverse; SEQ ID NO:2) versus AMPKα2-null [same forward primer as above] (forward) and [5′-GCATTGAACCACAGTCCTTCCTC-3′] (reverse; SEQ ID NO:3). PCR amplification yielded 200-bp fragments for WT and 600-bp fragments for null mice. All IOP measurements were taken between 6 and 7 weeks of age. The mouse iridocorneal angle and its structures reach maturity by 5 weeks.⁵⁵

Measurement of IOP

Mouse IOP was measured as previously described and validated.²⁶ Mice were anesthetized by intraperitoneal (IP) injection of a ketamine/xylazine mixture (100 mg/kg and 9 mg/kg, respectively; Phoenix Pharmaceutica, St. Joseph, Mo.). Per manufacturer recommendations, the rebound tonometer (TonoLab, Colonial Medical Supply, Franconia, N.H.) was fixed horizontally to allow perpendicular contact with the central cornea, and the tip of the probe was positioned between 2 and 3 mm from the eye. To reduce variability, the rebound tonometer was modified to include a pedal that activated the probe, obviating handling of the device. Target verification was performed under direct visualization at 5.5× magnification. A single measurement was accepted only if the device indicated that there was “no significant variability” (per protocol manual; Colonial Medical Supply). The average IOP was taken from three sets of six measurements of IOP in each eye, alternating right and left eye, with the starting eye picked at random.^{56, 57} All measurements were taken between 4 and 7 minutes after IP injection, as previous studies have shown this to be a period of stable IOP.^{58, 59} Previous studies have shown that weekly administration of this anesthesia mixture does not affect IOP.⁶⁰ IOP was measured once per mouse, between 11 am and 3 pm at 7 weeks of age—1 week after CCT measurement.

Optical Coherence Tomography

Eyes of adult mice (at 6 weeks) were imaged using optical coherence tomography (OCT) (Stratus; Carl Zeiss Meditec Inc.; Dublin, Calif.). Under general anesthesia by IP injection of a ketamine/xylazine mixture, mouse eyes were scanned to acquire images and were analyzed using OCT software (Stratus version 4.0.7; Carl Zeiss Meditec). CCT was determined by measuring the distance between 2 peaks representing the corneal epithelium and endothelium. Measurements were performed in triplicate for each eye by the same investigator who was masked to the mouse strain. Values were averaged and reported as means and standard deviations. We have previously validated the use of OCT in mice to estimate CCT against high-frequency ultrasound and histology.²⁶

Light Microscopy

For light microscopy, mice were euthanized using CO₂, and then immediately enucleated. The eyes were fixed with 10% formalin for 2 days, dehydrated in 70% Ethanol, then rehydrated in ascending concentrations of ethanol (70%, 95%, 100%) for 2 hours. The eyes were incubated with methacrylate (Technovit 7100, Heraeus Kulzer GmbH, Wehrheim, Germany) and Harder 1 and 2 (Technovit 7100, Heraeus Kulzer GmbH, Wehrheim, Germany) for 2 hours. Fixed sections were cut at 3 μm, and then stained with Toluidine Blue.

Assessment of Aqueous Humor Clearance

To investigate the mechanism of the observed IOP difference between AMPKα2-null mice and their WT counterparts, we noninvasively measured aqueous humor clearance using a modified approach to a previously published fluorophotometric technique.⁶¹ All measurements were made between 11 am and 3 pm, to reduce potential variability related to diurnal variation of aqueous inflow or outflow. After anesthetizing each mouse with the same solution used for IOP measurement, 10 μL of 0.02% benzalkonium chloride (BAC) in saline were applied to the right eye to permeabilize the cornea to fluorescein.⁶² After 5 minutes, the BAC solution was blotted at the lid margin without contacting the corneal epithelium and 10 μL of 0.02% fluorescein in saline was applied to the eye for 5 minutes. The eye and lids were then carefully washed with 600 μL saline. The microscope was focused to a depth intermediate to the iris and cornea, and images were captured in 10-minute intervals thereafter for 1 hour (AxioCam ICC 1 camera and Stemi SV11 microscope; Crl Zeiss Meditec, Inc.) Using ImageJ software, an area with no corneal defects was selected and analyzed for average pixel intensity in the green channel. All averages were normalized to the intensity calculated for the image taken at time 0.

Trabecular Meshwork Cell Culture

Primary human trabecular meshwork (TM) cells were isolated, in accordance with the Declaration of Helsinki, and maintained in culture as described previously.⁶³ Independent primary human TM cell lines were generated from donors ranging in age from 35 to 72 years and no known history of ocular disease. Cell cultures were maintained, unless otherwise stated, in Dulbecco's modified eagle medium (Life Technologies, Grand Island, N.Y.) containing 20% fetal bovine serum, 1% L-glutamine (2 mM), and gentamicin (0.1 mg/ml) at 37° C. in a 10% CO₂ atmosphere. Only TM cells from third through fifth passage were used. All experiments were performed using at least three different primary human TM cell lines.

Immunoblot Analysis

At the conclusion of each experiment for matricellular and ECM protein detection, conditioned media (CM) from TM cell cultures was harvested and centrifuged at 5000 rpm for 10 minutes at 4° C. The supernatant was then concentrated (Amicon Ultra-4 Filter Unit, 10 kDa; Millipore, Milford, Mass.), and protein content quantified using the DC Protein Assay kit adhering to manufacturer's protocols (Bio-Rad, Hercules, Calif.). For AMPK protein detection, cells were lysed for 3 minutes on ice with cold 1×RIPA buffer containing 0.5% Aprotinin, 0.1% EDTA, 1% EGTA, 0.5% PMSF, and 0.01% Leupeptin. Samples were then centrifuged at 14,000 rpm for 15 minutes at 4° C. and protein content quantified. In all experiments, equal amounts of protein were treated with 6× reducing buffer and boiled for 5 minutes. Samples were then electrophoresed in 10% SDS-polyacrylamide gels, alongside a pre-stained protein marker (Cell Signaling Technology Inc., Danvers, Ma). For conditioned media loading control, the resultant gels were stained with 0.1% Coomassie Brilliant Blue G-250 (Bio-Rad, Hercules Calif.) for 3 hours and were destained with fixing/destaining solution until clear bands were visible and contrasted well with the true blue background. Otherwise, proteins were transferred to nitrocellulose membranes (0.45-μm pore size; Invitrogen). Membranes were blocked for 1 hour at room temperature (RT) in a 1:1 mixture of 1×TBS-T (20 mM Tris-HCl [pH 7.6], 137 mM NaCl, 0.1% Tween-20) and blocking buffer (Rockland, Inc., Gilbertsville, Pa.), followed by overnight incubation at 4° C. with the indicated primary antibody at 1:10,000 for SPARC (Hematologic Technologies Inc., Essex Junction, Vt.), 1:1000 for TSP-1 (AF3074 R&D Systems Inc., Minneapolis, Minn.), 1:1000 for COL1 (600-401-103-0.5 Rockland Inc., Gilbertsville, Pa.), 1:1000 for COL4 (600-401-106-0.5 Rockland Inc., Gilbertsville, Pa.), 1:200 for Laminin (L8271 Sigma-Aldrich Inc., St. Louis, Mo.), and 1:000 for p(Thr172)-AMPKα, AMPKα, AMPKα1, AMPKα2, p-ACC, and ACC (Cell Signaling). A 1:200 dilution was used for p(Ser188)-RhoA and for total RhoA (Santa Cruz Biotechnology), and a 1:1000 dilution was used for Myc-Tag and for β-actin (Cell Signaling). Following incubation with primary antibody, the membranes were washed three times with 1×TBS-T and incubated for 1 hour at RT with dye-conjugated affinity purified 680 anti-mouse or 800 anti-rabbit IgG antibodies, respectively (IRDye; 1:10,000 dilution; Rockland Inc., Gilbertville, Pa.). The membranes were then washed three times with 1×TBS-T, scanned, and integrated band intensities were calculated using an infrared imaging system (Odyssey; Li-Cor, Lincoln, Nebr.).

Immunofluorescent Staining of Human Anterior Segments

Human donor eyes (aged 21, 44, 65, and 84) were immersion-fixed in 10% neutral buffered formalin within 15 hours of enucleation, dehydrated in sequential ethanol solutions (75%, 85%, 95%, 100%), and then embedded in paraffin. Sections (6 μm) were mounted on poly-L-lysine-coated glass slides and baked for 2 hours at 60° C. Slides were then deparaffinized in xylene, sequentially rehydrated in ethanol solutions, and washed three times for ten minutes in phosphate-buffered saline containing 0.1% Tween-20 (PBS-T). After one hour of incubation in 10% goat serum, tissues were permeabilized for 5 minutes in 0.2% Triton-100 in 1×PBS and washed three times in PBS-T. Prepared sections were incubated overnight at 4° C. in either primary AMPKα1 or AMPKα2 antibody diluted 1:200 in PBS or in PBS alone. Slides were washed three times in PBS-T and then incubated in 1:200 goat anti-rabbit 594 Alex Fluor secondary IgG (Invitrogen, Carlsbad, Calif.), followed by three additional washes. Nuclei were stained using DAPI antifade reagent (SlowFade Gold; Invitrogen). Labeled tissues were imaged and analyzed by fluorescent light microscopy using a Zeiss Observer3.1.

Immunofluorescent Staining of Primary Cultured Human TM Cells

TM cells in 8 well-slides were fixed for 30 minutes with 4% paraformaldehyde in PBS (pH 7.4) at 4° C., then washed in PBS for 10 minutes twice at RT. Cells were permeabilized with 0.2% Triton-100 in PBS for 5 min and then washed in PBS and blocked in 3% bovine serum albumin (BSA) in PBS for 1 hr at RT. Primary 568 phalloidin (F-actin) Alexa Fluor® antibody (Invitrogen) was applied at 1:100 dilution to each section and incubated overnight at 4° C. Slides were washed with 3% BSA-PBS for 10 min, 3 times. Nuclei were stained with TO-DAPI (Invitrogen), and labeled tissues were analyzed by fluorescent light microscopy using a Zeiss Observer3.1.

AICAR Time Course Experiments

TM cells at 90-100% confluency were cultured in serum-free media (SF) for 8 hours, and then incubated for the indicated time intervals in SF media containing 0.5 mM AICAR (Calbiochem, San Diego, Calif.) prior to lysis and immunoblot analysis as described above.

TGF-β2 Time Course Experiments

TM cells at 90-100% confluency were serum starved for 8 hours and then incubated in SF media containing 2.5 ng/mL activated TGF-β2 (R&D Systems, Minneapolis, Minn.) for the indicated time intervals prior to processing as above. Where noted, 4 mM HCl containing 0.1% human serum albumin served as the vehicle for TGF-β2.

Adenoviral-Mediated Infection Experiments

TM cells at 70-90% confluency were infected in 2% FBS media with either adenovirus expressing a dominant negative form of the AMPKα subunit (ad.DN.AMPKα) or control empty adenoviral vector (ad.null) at 25 MOI (Eton Bioscience, Charlestown, Mass.). MOI is the ratio of infectious units (viruses) to infection targets (cells).^{64,65} The ad.DN.AMPKα virus expresses an a2 subunit harboring a K45R mutation in the kinase domain, which competes for binding with the β and γ subunits but lacks kinase activity. After 18 hours, an equal volume of 10% FBS media was added to each well and cells were incubated for an additional 48 hours then lysed.

Effects of AICAR on IOP in Perfused Ex Vivo Human Anterior Segments

The effects of AICAR on IOP in perfused constant flow ex vivo human anterior segments was examined using well-established and validated methods (see, e.g., Oh et al., Invest. Ophthalmol. Vis. Sci. 2013; 54:3309-19; Ethier et al., Invest. Ophthalmol. Vis. Sci. 2004; 45:1863-70). Briefly, all donor pairs of eyes (aged 84, 71, and 82 years) were obtained from National Disease Research Interchange (NDRI, Philadelphia, Pa.) according to the provisions of the Declaration of Helsinki for research involving human tissue. Eyes were obtained within 24 hours after death. No donors were known to have a history of glaucoma or other ocular disorder. Human perfused anterior segment cultures were prepared by scoring the surface of the eye around ora serrata with a surgical blade, and the full-thickness incision was completed around the eye with scissors. The vitreous, lens, and iris were removed. Ciliary processes were dissected carefully, leaving in place the longitudinal portion of the ciliary muscle. The anterior segments were rinsed thoroughly with culture media and were mounted into custom plexiglass culture chambers. Anterior segments were perfused at a constant flow rate of 2.5 μL/min with DMEM (Invitrogen-Gibco) containing 1% FBS, 1% L-glutamine (2 mM), penicillin (100 U/mL), streptomycin (100 U/mL), gentamicin (0.17 mg/mL), and amphotericin-B (0.25 μg/mL) under 5% CO₂ at 37° C., using microinfusion pumps (Harvard Apparatus, Holliston, Mass.). IOP was monitored with a pressure transducer (Argon Medical Devices, Athens, Tex.) and were recorded with an automated computerized system (National Instruments, Austin, Tex.) every second and averaged each hour. Perfused tissue was allowed to equilibrate at 37° C. and 5% CO2 until a stable baseline IOP was achieved, typically 2 to 4 days. Then one eye was perfused with 2.5 μl of 1×PBS per 1 mL of ex vivo media as a control while the opposite eye received 2.5 μL of 200 mM AICAR per 1 mL of ex vivo media. The chambers were kept in a 5% CO2, 37° C. humidified incubator. Effects of AICAR treatment on IOP are expressed as the percentage change in IOP (compared to baseline). Values are expressed as mean±SEM, and paired two-tailed student t-tests are applied to determine significance of difference in IOP between control and experimental groups at selected time intervals. IOP is normalized at time point 0, the time of initial treatment.

Statistics

GraphPad Prism 6 software (GraphPad, La Jolla, Calif.) was used. A two-tailed student t-test was used for comparing differences between two groups, and differences were considered significant when p<0.05. Throughout, n refers to the number of independent experiments performed using different primary human TM cell lines, established from separate donors.

### Example 1

### AMPKα2-Null Mice Exhibit Increased IOP and Decreased Aqueous Humor Clearance

AMPKα2-null mice exhibited a 6% higher IOP (p=0.0265) than WT counterparts (FIG. 1A), with no significant difference (p=0.6877) in CCT (FIG. 1B). AMPKα2-null mice had a mean IOP of 18.2±0.28 mmHg versus the WT mean IOP of 17.2±0.36 mmHg. By light microscopy, the iridocorneal angles in AMPKα2-null mice appeared grossly indistinguishable from WT counterparts with similar outflow structures and cellularity (FIG. 1C).

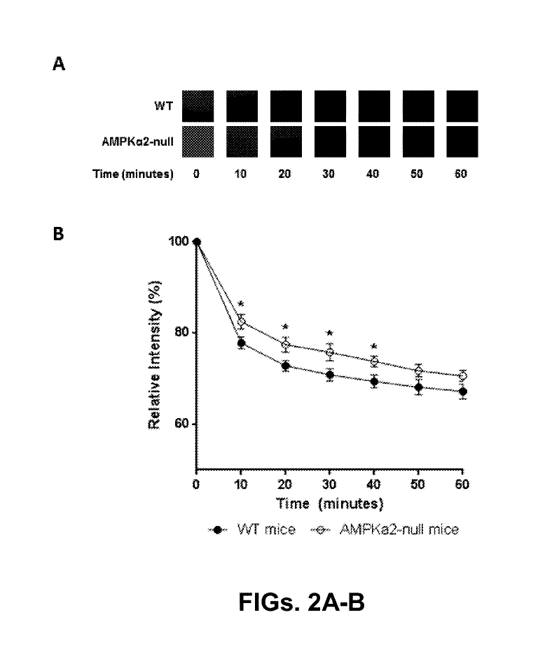

Aqueous humor clearance in AMPKα2-null mice was reduced compared to their WT counterparts (FIG. 2). Least-squares fit analysis yielded exponential decay constants of 0.1112%/min (r²=0.91) and 0.0854%/min (r²=0.91) for WT and AMPKα2-null mice, respectively. Fluorescent intensities were greater at each time point for AMPKα2-null, and student t-tests revealed significant differences between AMPKα2-null and WT at 10 minutes, 20 minutes, 30 minutes, and 40 minutes (p<0.05).

### Example 2

### AMPKα1 and AMPKα2 Isoforms are Expressed in Human TM and AICAR Treatment Leads to Activation

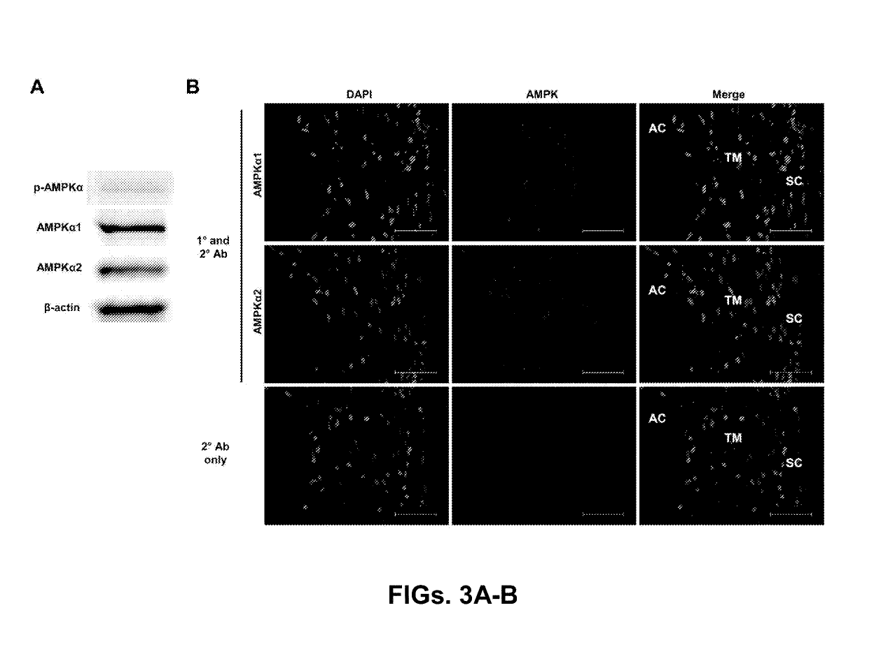

In its active form, AMPK exists as a heterotrimer with two regulatory β and γ subunits joined with a catalytic α subunit that has two distinct isoforms (α1 and α2).³³ Both isoforms were detectable by immublot (FIG. 3A) Immunofluorescent microscopy revealed that both isoforms were prominent in the TM, lining the trabecular beams and inner and outer walls of Schlemm's canal (FIG. 3B).

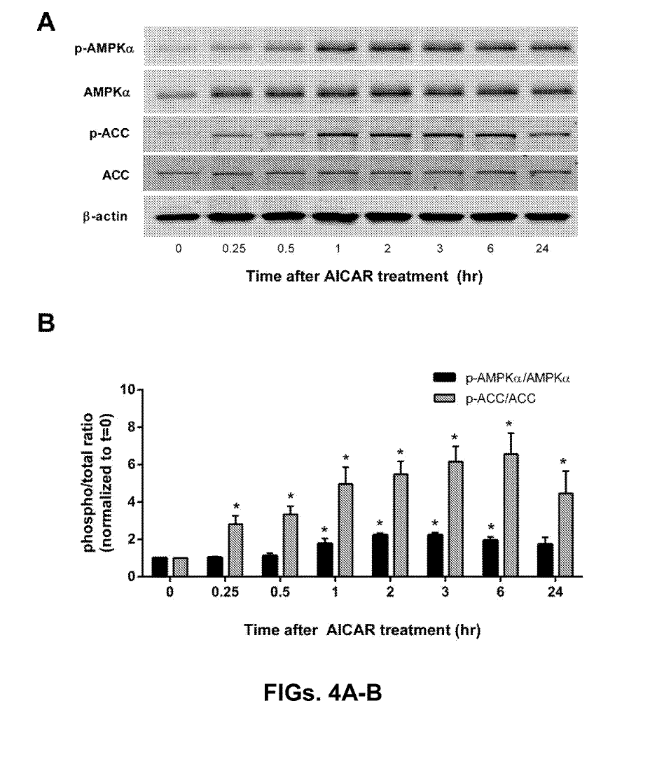

An adenosine analog, 5-Aminoimidazole-4-carboxamide riboside (AICAR) reproduces the effects of extracellular AMP and activates AMPKα via increased phosphorylation at Thr172.^{40, 41, 66} To examine whether AICAR phosphorylates and activates AMPKα in the TM, TM cells were incubated with 0.5 mM AICAR for a 24-hour time course and then lysed for immunoblot analysis (FIG. 4A). The ratio of phospho-total AMPK, normalized to the zero time point, increased by 77% within 1 hour of AICAR treatment (p=0.0323) and peaked with a greater than 2-fold increased ratio at 2 hours (FIG. 4B). To determine whether phosphorylation of AMPK led to functional activation, the phospho-total ratio of the known downstream signaling target Acetyl-CoA carboxylase (ACC)³⁴ was similarly analyzed (FIG. 4A, 4B). The phospho-total ACC ratio increased by a statistically significant degree within 15 minutes (p=0.0076), peaking with a 6.6-fold increased ratio at 6 hours.

### Example 3

### AICAR Suppresses ECM Proteins and Alters Cytoskeleton in TM Under Basal and TGF-β2 Stimulatory Conditions

To examine whether modulation of AMPK affects certain matricellular and ECM proteins, TM cells were treated with 0.5 mM AICAR or PBS vehicle and CM was probed for SPARC, TSP-1, collagen I, collagen IV, and laminin (FIG. 5A). Calculation of mean integrated band intensities revealed 70%, 52%, and 64% decreases in collagen I, collagen IV, and laminin, respectively (p<0.001) but no change in SPARC or TSP-1 (FIG. 5B). Under TGF-β2 stimulation with 2.5 ng/mL, AICAR treatment decreased SPARC, collagen I, collagen IV, and laminin levels by 64%, 26%, 34%, and 33%, respectively (p<0.001) with no change in TSP-1 levels (FIG. 5C, 5D).

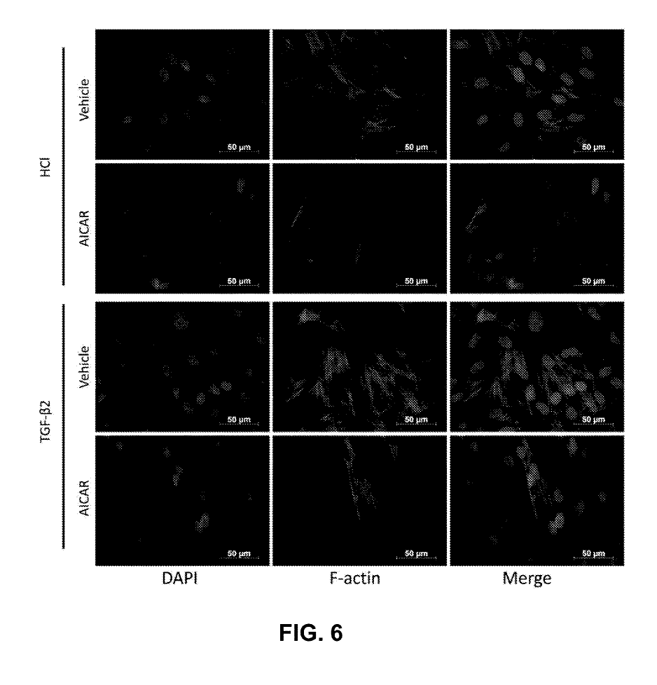

Evidence suggests that cellular tone within the TM can contribute to outflow facility.^{19, 20} Cultured TM cells treated with 0.5 mM AICAR displayed narrower cell bodies (data not shown). Under basal and TGF-β2 stimulatory conditions, AICAR-treated cells exhibited decreased F-actin staining and actin stress fiber formation (FIG. 6).

### Example 4

### AICAR Treatment Leads to Phosphorylation of RhoA at Ser188

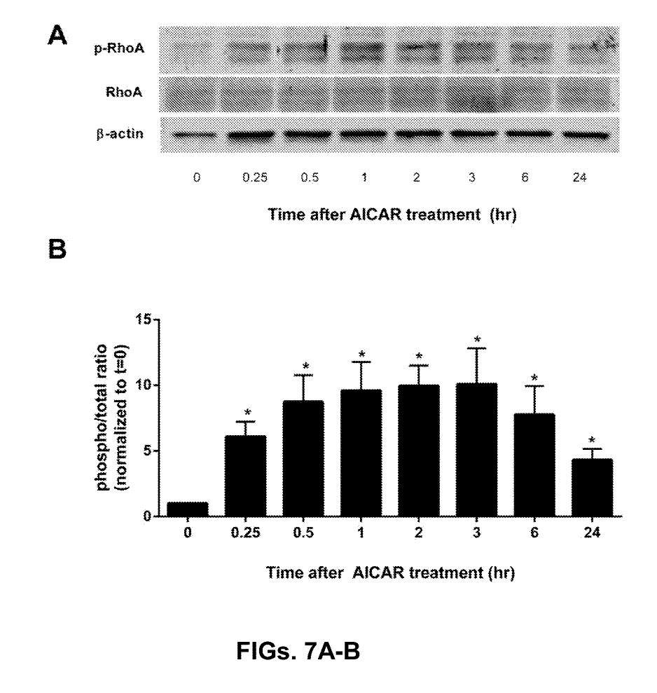

RhoA induces ECM deposition in TM cells, contributing to increased resistance to aqueous humor outflow.^{19, 20} Phosphorylation of RhoA at Ser188 uncouples the RhoA/RhoA-associated protein kinase (ROCK) pathway that mediates increased ECM deposition.^{67, 68} A recent study demonstrated that activated AMPK directly phosphorylates RhoA at Ser188.⁶⁹ When TM cells were treated with AICAR, the phosphototal RhoA ratio increased approximately 10-fold within one hour and remained statistically significant through 24 hours (FIG. 7).

### Example 5

### TGF-β2 Treatment Leads to Transient Dephosphorylation of AMPKα in TM

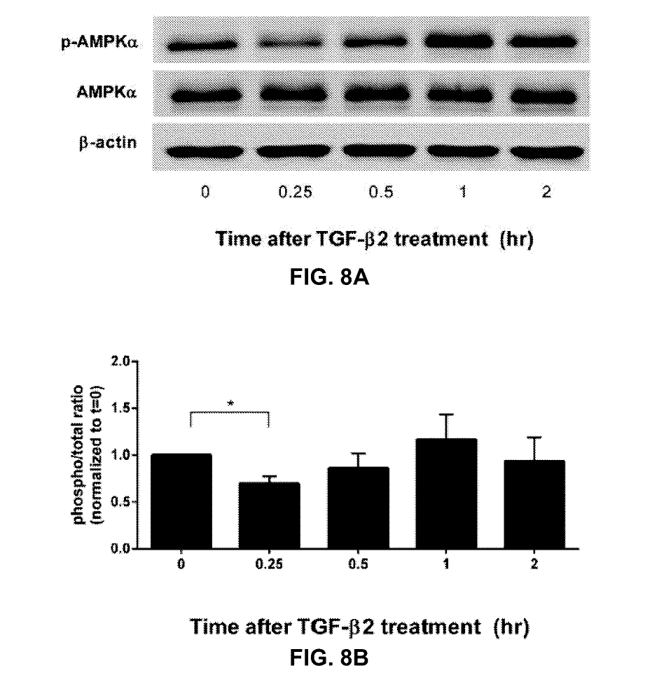

To assess whether TGF-β2 has an effect on AMPK signaling, TM cells were incubated with 2.5 ng/mL TGF-β2. The phospho-total AMPK ratio was calculated and normalized to the zero time point (FIG. 8). The ratio decreased by 30% within 15 minutes (p=0.0067), but the difference was no longer significant at 30 minutes, returning to baseline.

### Example 6

### Adenoviral Transfer of Dominant Negative AMPKα Induces ECM Expression in TM

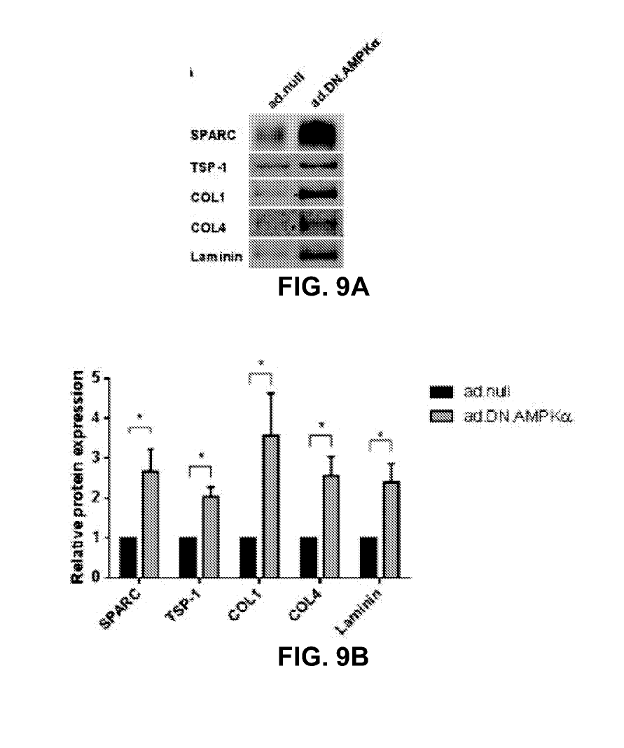

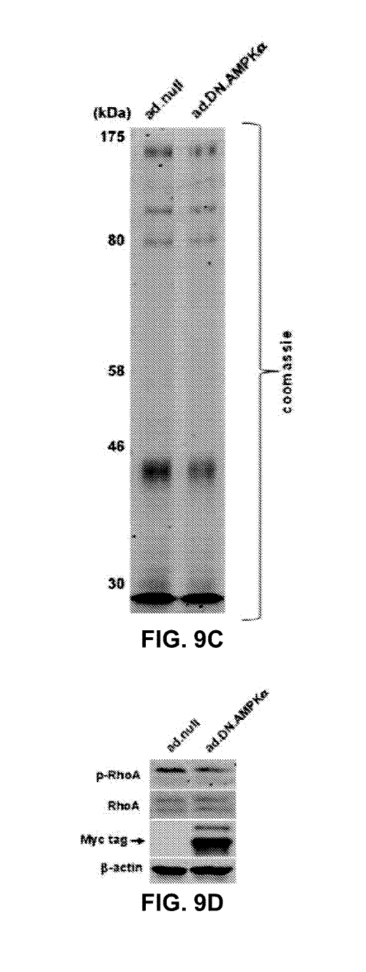

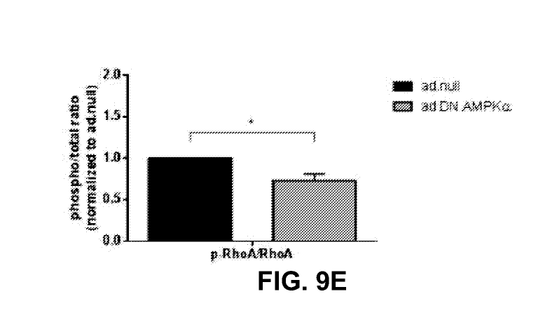

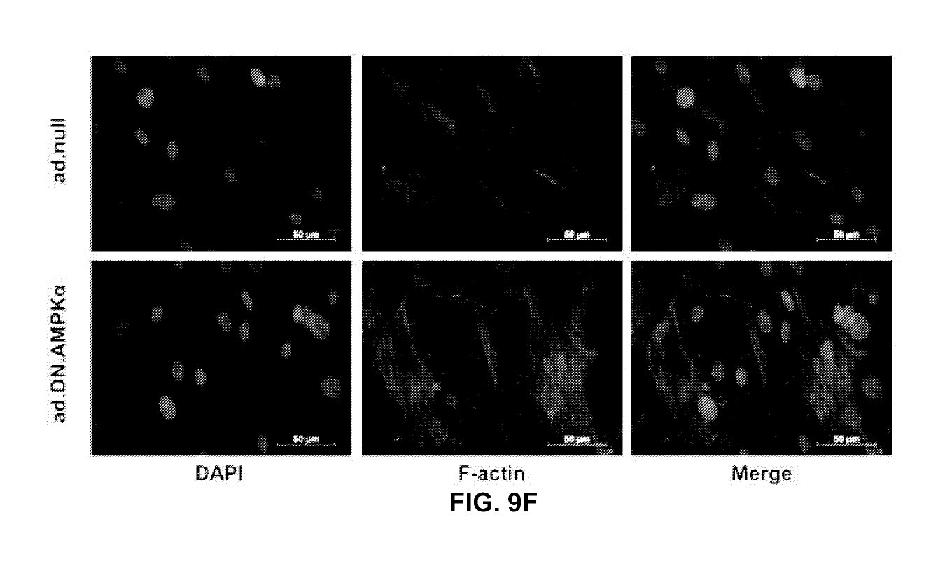

In the CM of cells expressing the dominant negative AMPKα subunit SPARC, TSP-1, collagen I, collagen IV, and laminin protein levels were increased 2.7-fold (p=0.0137), 2.0-fold (p=0.0012), 3.6-fold (p=0.0497), 2.6-fold (p=0.0178), and 2.4-fold (p=0.0239), respectively (FIG. 9). A 27% decrease in the phospho-total RhoA ratio (p=0.0053) was observed in the corresponding cell lysates (FIG. 9). Additionally, transfer of ad.DN.AMPKα led to marked increases in F-actin cytoskeletal staining and cytoskeletal disarray (FIG. 9E).

### Example 7

### Effects of AICAR on IOP in Perfused Ex Vivo Human Anterior Segments

The effects of AICAR on IOP in perfused constant flow ex vivo human anterior segments was examined using established and validated methods (Oh et al., Invest. Ophthalmol. Vis. Sci. 2013; 54:3309-19; Ethier et al., Invest. Ophthalmol. Vis. Sci. 2004; 45:1863-70).

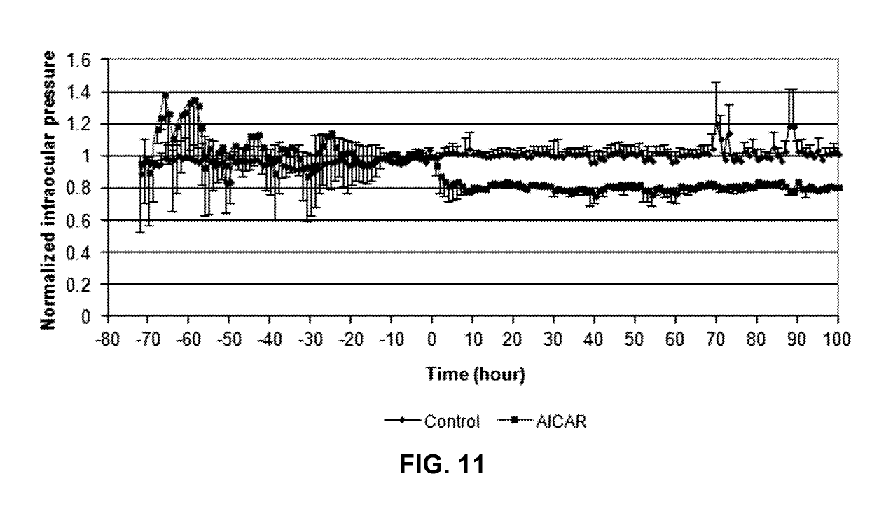

Briefly, all donor pairs of eyes (aged 84, 71, and 82 years) are obtained within 24 hours after death from National Disease Research Interchange (NDRI, Philadelphia, Pa.) according to the provisions of the Declaration of Helsinki for research involving human tissue. Donors with a known history of glaucoma or other ocular disorder were excluded. Treatment of constant-flow-perfused ex vivo human anterior segments as described above with 2.5 μL of 200 mM AICAR (in 1 mL of ex vivo media) resulted in a mean decrease in IOP of 18.54±1.78% by day 7, compared with paired opposite eye controls (FIG. 11). Data expressed as mean percentage change in IOP±SEM (p<0.05 for paired t-tests; n=3).

### Example 8

### Effects of AMPK Activators on Ocular Hypertensive NZW Rabbits

To elucidate further the role of AMPK signaling in regulating IOP, the effects of topical administration of these agents in ocular hypertensive New Zealand White (NZW) rabbits are assessed using methods similar to those performed in a previous study of novel IOP-modulating agents.⁹⁵ For each rabbit IOP measurement, eyes are first anesthetized by topical application of 0.4% oxybuprocaine. An applanation tonometer (Tono-Pen; Medtronic Solan, Jacksonville, Fla.) is then calibrated and recordings averaged over three measurements. Prior to induction of ocular hypertension, baseline IOP is recorded bilaterally. Following anesthetization by intramuscular injection of 50 mg/mL ketamine and 2% Rompun, a 26 gauge needle is used to create a temporal paracentesis and a 27 gauge needle is used to inject 50 μL Viscoat (Alcon Laboratories, Fort Worth, Tex.) into the anterior chamber. The paracentesis is then hydrated with saline to prevent reflux of aqueous humor. 0.5 mM AICAR versus PBS vehicle is administered topically three times at 0, 3, and 6 hours post Viscoat injection. IOP is measured every hour for 8 hours. Data is analyzed using Student's t-test for individual time points. The IOP time course is analyzed using ANOVA for repeated measurements (GraphPad Prism 5.0; GraphPad Software, Inc., San Diego, Calif.). Data is presented as mean±SEM and p-values less than 0.05 will be considered statistically significant.

### REFERENCES

1. Nemesure B, Honkanen R, Hennis A, Wu S Y, Leske M C, Barbados Eye Studies Group. Incident open-angle glaucoma and intraocular pressure. Ophthalmology 2007; 114:1810-1815.
2. Larsson L I, Rettig E S, Brubaker R F. Aqueous flow in open-angle glaucoma. Arch. Ophthalmol. 1995; 113:283-286.
3. [Anonymous]. The advanced glaucoma intervention study (AGIS): 7. the relationship between control of intraocular pressure and visual field deterioration.the AGIS investigators. Am. J. Ophthalmol. 2000; 130:429-440.
4. Heijl A, Leske M C, Bengtsson B, et al. Reduction of intraocular pressure and glaucoma progression: Results from the early manifest glaucoma trial. Arch. Ophthalmol. 2002; 120:1268-1279.
5. Allingham R R, Liu Y, Rhee D J. The genetics of primary open-angle glaucoma: A review. Exp. Eye Res. 2009; 88:837-844.
6. Pederson J E, Gaasterland D E, MacLellan H M. Uveoscleral aqueous outflow in the rhesus monkey: Importance of uveal reabsorption. Invest. Ophthalmol. Vis. Sci. 1977; 16:1008-1007.
7. Lei Y, Overby D R, Boussommier-Calleja A, Stamer W D, Ethier C R. Outflow physiology of the mouse eye: Pressure dependence and washout. Invest. Ophthalmol. Vis. Sci. 2011; 52:1865-1871.
8. Aihara M, Lindsey J D, Weinreb R N. Aqueous humor dynamics in mice. Invest. Ophthalmol. Vis. Sci. 2003; 44:5168-5173.
9. Bill A. Editorial: The drainage of aqueous humor. Invest. Ophthalmol. 1975; 14:1-3.
10. Seiler T, Wollensak J. The resistance of the trabecular meshwork to aqueous humor outflow. Graefes Arch. Clin. Exp. Ophthalmol. 1985; 223:88-91.
11. Acott T S, Kingsley P D, Samples J R, Van Buskirk E M. Human trabecular meshwork organ culture: Morphology and glycosaminoglycan synthesis. Invest. Ophthalmol. Vis. Sci. 1988; 29:90-100.
12. Knepper P A, Farbman A I, Telser A G. Exogenous hyaluronidases and degradation of hyaluronic acid in the rabbit eye. Invest. Ophthalmol. Vis. Sci. 1984; 25:286-293.
13. Barany E H, Scotchbrook S. Influence of testicular hyaluronidase on the resistance to flow through the angle of the anterior chamber. Acta Physiol. Scand. 1954; 30:240-248.
14. Bradley J M, Vranka J, Colvis C M, et al. Effect of matrix metalloproteinases activity on outflow in perfused human organ culture. Invest. Ophthalmol. Vis. Sci. 1998; 39:2649-2658.
15. Keller K E, Aga M, Bradley J M, Kelley M J, Acott T S. Extracellular matrix turnover and outflow resistance. Exp. Eye Res. 2009; 88:676-682.
16. Fuchshofer R, Tamm E R. Modulation of extracellular matrix turnover in the trabecular meshwork. Exp. Eye Res. 2009; 88:683-688.
17. Liton P B, Gonzalez P, Epstein D L. The role of proteolytic cellular systems in trabecular meshwork homeostasis. Exp. Eye Res. 2009; 88:724-728.
18. Tian B, Gabelt B T, Geiger B, Kaufman P L. The role of the actomyosin system in regulating trabecular fluid outflow. Exp. Eye Res. 2009; 88:713-717.
19. Zhang M, Maddala R, Rao P V. Novel molecular insights into RhoA GTPase-induced resistance to aqueous humor outflow through the trabecular meshwork. Am. J. Physiol. Cell. Physiol. 2008; 295:C1057-70.
20. Pattabiraman P P, Rao P V. Mechanistic basis of rho GTPase-induced extracellular matrix synthesis in trabecular meshwork cells. Am. J. Physiol. Cell. Physiol. 2010; 298:C749-63.
21. Rohen J W. Presence of matrix vesicles in the trabecular meshwork of glaucomatous eyes. Graefes Arch. Clin. Exp. Ophthalmol. 1982; 218:171-176.
22. Stamer W D. The cell and molecular biology of glaucoma: Mechanisms in the conventional outflow pathway. Invest. Ophthalmol. Vis. Sci. 2012; 53:2470-2472.
23. Yan Q, Clark J I, Sage E H. Expression and characterization of SPARC in human lens and in the aqueous and vitreous humors. Exp. Eye Res. 2000; 71:81-90.
24. Rhee D J, Fariss R N, Brekken R, Sage E H, Russell P. The matricellular protein SPARC is expressed in human trabecular meshwork. Exp. Eye Res. 2003; 77:601-607.
25. Oh D J, Kang M H, Ooi Y H, Choi K R, Sage E H, Rhee D J. Overexpression of SPARC in human trabecular meshwork increases intraocular pressure and alters extracellular matrix. Invest. Ophthalmol. Vis. Sci. 2013.
26. Haddadin R I, Oh D J, Kang M H, et al. SPARC-null mice exhibit lower intraocular pressures. Invest. Ophthalmol. Vis. Sci. 2009; 50:3771-3777.
27. Swaminathan S S, Oh D J, Kang M H, et al. Secreted protein, acidic and rich in cysteine (SPARC)-null mice exhibit more uniform outflow. Invest. Ophthalmol. Vis. Sci. 2013.
28. Rhee D J, Haddadin R I, Kang M H, Oh D J. Matricellular proteins in the trabecular meshwork. Exp. Eye Res. 2009; 88:694-703.
29. Flugel-Koch C, Ohlmann A, Fuchshofer R, Welge-Lussen U, Tamm E R. Thrombospondin-1 in the trabecular meshwork: Localization in normal and glaucomatous eyes, and induction by TGF-beta1 and dexamethasone in vitro. Exp. Eye Res. 2004; 79:649-663.
30. Haddadin R I, Oh D J, Kang M H, et al. Thrombospondin-1 (TSP1)-null and TSP2-null mice exhibit lower intraocular pressures. Invest. Ophthalmol. Vis. Sci. 2012; 53:6708-6717.
31. Motoshima H, Goldstein B J, Igata M, Araki E. AMPK and cell proliferation—AMPK as a therapeutic target for atherosclerosis and cancer. J. Physiol. 2006; 574:63-71.
32. Viollet B, Horman S, Leclerc J, et al. AMPK inhibition in health and disease. Crit. Rev. Biochem. Hol. Biol. 2010; 45:276-295.
33. Viollet B, Athea Y, Mounier R, et al. AMPK: Lessons from transgenic and knockout animals. Front. Biosci. 2009; 14:19-44.
34. Hardie D G, Ross F A, Hawley S A. AMP-activated protein kinase: A target for drugs both ancient and modern. Chem. Biol. 2012; 19:1222-1236.
35. Barnes B R, Zierath J R. Role of AMP-activated protein kinase in the control of glucose homeostasis. Curr. Mol. Med. 2005; 5:341-348.
36. Li M, Verdijk L B, Sakamoto K, Ely B, van Loon L J, Musi N. Reduced AMPK-ACC and mTOR signaling in muscle from older men, and effect of resistance exercise. Mech. Ageing Dev. 2012; 133:655-664.
37. Durante P E, Mustard K J, Park S H, Winder W W, Hardie D G. Effects of endurance training on activity and expression of AMP-activated protein kinase isoforms in rat muscles. Am. J. Physiol. Endocrinol. Metab. 2002; 283:E178-86.
38. Park S H, Paulsen S R, Gammon S R, Mustard K J, Hardie D G, Winder W W. Effects of thyroid state on AMP-activated protein kinase and acetyl-CoA carboxylase expression in muscle. J. Appl. Physiol. 2002; 93:2081-2088.
39. Qiang W, Weiqiang K, Qing Z, Pengju Z, Yi L. Aging impairs insulin-stimulated glucose uptake in rat skeletal muscle via suppressing AMPKalpha. Exp. Mol. Med. 2007; 39:535-543.
40. Cacicedo J M, Benjachareonwong S, Chou E, Yagihashi N, Ruderman N B, Ido Y. Activation of AMP-activated protein kinase prevents lipotoxicity in retinal pericytes. Invest. Ophthalmol. Vis. Sci. 2011; 52:3630-3639.
41. Kubota S, Ozawa Y, Kurihara T, et al. Roles of AMP-activated protein kinase in diabetes-induced retinal inflammation. Invest. Ophthalmol. Vis. Sci. 2011; 52:9142-9148.
42. Omae T, Nagaoka T, Tanano I, Yoshida A. Pioglitazone, a peroxisome proliferator-activated receptor-gamma agonist, induces dilation of isolated porcine retinal arterioles: Role of nitric oxide and potassium channels. Invest. Ophthalmol. Vis. Sci. 2011; 52:6749-6756.
43. Tripathi R C, Li J, Chan W F, Tripathi B J. Aqueous humor in glaucomatous eyes contains an increased level of TGF-beta 2. Exp. Eye Res. 1994; 59:723-727.
44. Yamamoto N, Itonaga K, Marunouchi T, Majima K. Concentration of transforming growth factor beta2 in aqueous humor. Ophthalmic Res. 2005; 37:29-33.
45. Ochiai Y, Ochiai H. Higher concentration of transforming growth factor-beta in aqueous humor of glaucomatous eyes and diabetic eyes. Jpn. J. Ophthalmol. 2002; 46:249-253.
46. Fleenor D L, Shepard A R, Hellberg P E, Jacobson N, Pang I H, Clark A F. TGFbeta2-induced changes in human trabecular meshwork: Implications for intraocular pressure. Invest. Ophthalmol. Vis. Sci. 2006; 47:226-234.
47. Bollinger K E, Crabb J S, Yuan X, Putliwala T, Clark A F, Crabb J W. Quantitative proteomics: TGFbeta(2) signaling in trabecular meshwork cells. Invest. Ophthalmol. Vis. Sci. 2011; 52:8287-8294.
48. Kang M H, Oh D J, Kang J H, Rhee D J. Regulation of SPARC by transforming growth factor beta-2 in human trabecular meshwork. Invest. Ophthalmol. Vis. Sci. 2013.
49. Lim J Y, Oh M A, Kim W H, Sohn H Y, Park S I. AMP-activated protein kinase inhibits TGF-beta-induced fibrogenic responses of hepatic stellate cells by targeting transcriptional coactivator p300. J. Cell. Physiol. 2012; 227:1081-1089.
50. Sasaki H, Asanuma H, Fujita M, et al. Metformin prevents progression of heart failure in dogs: Role of AMP-activated protein kinase. Circulation 2009; 119:2568-2577.
51. Myerburg M M, King J D, Jr, Oyster N M, et al. AMPK agonists ameliorate sodium and fluid transport and inflammation in cystic fibrosis airway epithelial cells. Am. J. Respir. Cell Mol. Biol. 2010; 42:676-684.
52. Park C S, Bang B R, Kwon H S, et al. Metformin reduces airway inflammation and remodeling via activation of AMP-activated protein kinase. Biochem. Pharmacol. 2012; 84:1660-1670.
53. Mizutani J, Tokuda H, Matsushima-Nishiwaki R, et al. Involvement of AMP-activated protein kinase in TGF-beta-stimulated VEGF synthesis in osteoblasts. Int. J. Mol. Med. 2012; 29:550-556.
54. Viollet B, Andreelli F, Jorgensen S B, et al. The AMP-activated protein kinase alpha2 catalytic subunit controls whole-body insulin sensitivity. J. Clin. Invest. 2003; 111:91-98.
55. Smith R S, Zabaleta A, Savinova O V, John S W. The mouse anterior chamber angle and trabecular meshwork develop without cell death. BMC Dev. Biol. 2001; 1:3.
56. Wang W H, Millar J C, Pang I H, Wax M B, Clark A F. Noninvasive measurement of rodent intraocular pressure with a rebound tonometer. Invest. Ophthalmol. Vis. Sci. 2005; 46:4617-4621.
57. Saeki T, Aihara M, Ohashi M, Araie M. The efficacy of TonoLab in detecting physiological and pharmacological changes of mouse intraocular pressure-comparison with TonoPen and microneedle manometery. Curr. Eye Res. 2008; 33:247-252.
58. Savinova O V, Sugiyama F, Martin J E, et al. Intraocular pressure in genetically distinct mice: An update and strain survey. BMC Genet. 2001; 2:12.
59. Aihara M, Lindsey J D, Weinreb R N. Twenty-four-hour pattern of mouse intraocular pressure. Exp. Eye Res. 2003; 77:681-686.
60. Danias J, Kontiola A I, Filippopoulos T, Mittag T. Method for the noninvasive measurement of intraocular pressure in mice. Invest. Ophthalmol. Vis. Sci. 2003; 44:1138-1141.
61. Avila M Y, Mitchell C H, Stone R A, Civan M M. Noninvasive assessment of aqueous humor turnover in the mouse eye. Invest. Ophthalmol. Vis. Sci. 2003; 44:722-727.
62. Burstein N L. Preservative alteration of corneal permeability in humans and rabbits. Invest. Ophthalmol. Vis. Sci. 1984; 25:1453-1457.
63. Oh D J, Martin J L, Williams A J, et al. Analysis of expression of matrix metalloproteinases and tissue inhibitors of metalloproteinases in human ciliary body after latanoprost. Invest. Ophthalmol. Vis. Sci. 2006; 47:953-963.
64. Mocanu J D, Yip K W, Alajez N M, et al. Imaging the modulation of adenoviral kinetics and biodistribution for cancer gene therapy. Mol. Ther. 2007; 15:921-929.
65. Mesonero A, Suarez D L, van Santen E, Tang D C, Toro H. Avian influenza in ovo vaccination with replication defective recombinant adenovirus in chickens: Vaccine potency, antibody persistence, and maternal antibody transfer. Avian Dis. 2011; 55:285-292.
66. Corton J M, Gillespie J G, Hawley S A, Hardie D G. 5-aminoimidazole-4-carboxamide ribonucleoside. A specific method for activating AMP-activated protein kinase in intact cells? Eur. J. Biochem. 1995; 229:558-565.
67. Sawada N, Itoh H, Miyashita K, et al. Cyclic GMP kinase and RhoA Ser188 phosphorylation integrate pro- and antifibrotic signals in blood vessels. Mol. Cell. Biol. 2009; 29:6018-6032.
68. Rolli-Derkinderen M, Sauzeau V, Boyer L, et al. Phosphorylation of serine 188 protects RhoA from ubiquitin/proteasome-mediated degradation in vascular smooth muscle cells. Circ. Res. 2005; 96:1152-1160.
69. Gayard M, Guilluy C, Rousselle A, et al. AMPK alpha 1-induced RhoA phosphorylation mediates vasoprotective effect of estradiol. Arterioscler. Thromb. Vasc. Biol. 2011; 31:2634-2642.
70. Millar J C, Clark A F, Pang I H. Assessment of aqueous humor dynamics in the mouse by a novel method of constant-flow infusion. Invest. Ophthalmol. Vis. Sci. 2011; 52:685-694.
71. Hall A. Rho family GTPases. Biochem. Soc. Trans. 2012; 40:1378-1382.
72. Emerson R O, Sage E H, Ghosh J G, Clark J I. Chaperone-like activity revealed in the matricellular protein SPARC. J. Cell. Biochem. 2006; 98:701-705.
73. Martinek N, Shahab J, Sodek J, Ringuette M. Is SPARC an evolutionarily conserved collagen chaperone? J. Dent. Res. 2007; 86:296-305.
74. McCurdy S, Baicu C F, Heymans S, Bradshaw A D. Cardiac extracellular matrix remodeling: Fibrillar collagens and secreted protein acidic and rich in cysteine (SPARC). J. Mol. Cell. Cardiol. 2010; 48:544-549.
75. Chlenski A, Guerrero L J, Salwen H R, et al. Secreted protein acidic and rich in cysteine is a matrix scavenger chaperone. PLoS One 2011; 6:e23880.
76. Warden S M, Richardson C, O'Donnell J, Jr, Stapleton D, Kemp B E, Witters L A. Post-translational modifications of the beta-1 subunit of AMP-activated protein kinase affect enzyme activity and cellular localization. Biochem. J. 2001; 354:275-283.
77. Gregor M, Zeold A, Oehler S, et al. Plectin scaffolds recruit energy-controlling AMP-activated protein kinase (AMPK) in differentiated myofibres. J. Cell. Sci. 2006; 119:1864-1875.
78. David R, Jenkins T Genetic markers in glaucoma. Br. J. Ophthalmol. 1980; 64:227-231.
79. Sheffield V C, Stone E M, Alward W L, et al. Genetic linkage of familial open angle glaucoma to chromosome 1q21-q31. Nat. Genet. 1993; 4:47-50.
80. Wiggs J L, Haines J L, Paglinauan C, Fine A, Sporn C, Lou D. Genetic linkage of autosomal dominant juvenile glaucoma to 1q21-q31 in three affected pedigrees. Genomics 1994; 21:299-303.
81. Wiggs J L, Del Bono E A, Schuman J S, Hutchinson B T, Walton D S. Clinical features of five pedigrees genetically linked to the juvenile glaucoma locus on chromosome 1q21-q31. Ophthalmology 1995; 102:1782-1789.
82. Wojciechowski R, Stambolian D, Ciner E, Ibay G, Holmes T N, Bailey-Wilson J E. Genomewide linkage scans for ocular refraction and meta-analysis of four populations in the myopia family study. Invest. Ophthalmol. Vis. Sci. 2009; 50:2024-2032.
83. Naiglin L, Gazagne C, Dallongeville F, et al. A genome wide scan for familial high myopia suggests a novel locus on chromosome 7q36. J. Med. Genet. 2002; 39:118-124.
84. Andersen J S, Pralea A M, DelBono E A, et al. A gene responsible for the pigment dispersion syndrome maps to chromosome 7q35-q36. Arch. Ophthalmol. 1997; 115:384-388.
85. Sotirova V N, Irkec M, Percin E F, Bladow K M, Damji K F, Sarfarazi M. Molecular genetic study of families with pseudoexfoliation syndrome (PEX) suggests two putative locations on 2p14-2cen and 2q35-q36 regions. Iovs 1999; 40:512.
86. Hitchings R A, Grierson I. Clinico pathological correlation in eyes with failed fistulizing surgery. Trans. Ophthalmol. Soc. U.K. 1983; 103 (Pt 1):84-88.
87. Miller M H, Joseph N H, Ennis K W, Grierson I, Hitchings R A. An animal model of filtration surgery. Trans. Ophthalmol. Soc. U.K. 1985; 104 (Pt 8):893-897.
88. Miller M H, Grierson I, Unger W I, Hitchings R A. Wound healing in an animal model of glaucoma fistulizing surgery in the rabbit. Ophthalmic Surg. 1989; 20:350-357.
89. Picht G, Welge-Luessen U, Grehn F, Lutjen-Drecoll E. Transforming growth factor beta 2 levels in the aqueous humor in different types of glaucoma and the relation to filtering bleb development. Graefes Arch. Clin. Exp. Ophthalmol. 2001; 239:199-207.
90. Hawley S A, Fullerton M D, Ross F A, et al. The ancient drug salicylate directly activates AMP-activated protein kinase. Science 2012; 336:918-922.
91. Keller K E, Vranka J A, Haddadin R I, et al. The effects of tenascin C knockdown on trabecular meshwork outflow resistance. Invest. Ophthalmol. Vis. Sci. 2013; 54:5613-5623.
92. Kim J, Ahn J H, Kim J H, et al. Fenofibrate regulates retinal endothelial cell survival through the AMPK signal transduction pathway. Exp. Eye Res. 2007; 84:886-893.
93. Omae T, Nagaoka T, Tanano I, Kamiya T, Yoshida A. Fenofibrate, an anti-dyslipidemia drug, elicits the dilation of isolated porcine retinal arterioles: Role of nitric oxide and AMP-activated protein kinase. Invest. Ophthalmol. Vis. Sci. 2012; 53:2880-2886.
94. Buys E S, Ko Y C, Alt C, et al. Soluble guanylate cyclase alpha1-deficient mice: A novel murine model for primary open angle glaucoma. PLoS One 2013; 8:e60156.
95. Van de Velde S, Van Bergen T, Sijnave D, et al. AMA0076, a novel, locally acting rho kinase inhibitor, potently lowers intraocular pressure in new zealand white rabbits with minimal hyperemia. Invest. Ophthalmol. Vis. Sci. 2014; 55:1006-1016.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of reducing intraocular pressure (IOP) in a mammal, the method comprising:
identifying a mammal in need of reduced IOP; and
administering to the mammal an effective amount of an amp-activated protein kinase (AMPK) activator sufficient to reduce IOP in the mammal.

2. A method of treating glaucoma in a mammal, the method comprising:
identifying a mammal who has glaucoma; and
administering to the mammal a therapeutically effective amount of an amp-activated protein kinase (AMPK) activator.

3. (canceled)

4. (canceled)

5. The method of claim 1, wherein the mammal has ocular hypertension, a primary or secondary form of acute or chronic open-angle glaucoma, a primary or secondary acute or chronic angle-closure glaucoma, and/or a congenital or developmental glaucoma.

6. The method of claim 1, wherein the AMPK activator is selected from the group consisting of 5-Aminoimidazole-4-carboxamide riboside (AICA riboside or AICAR); AICA ribotide (ZMP); guanidine; galegine; metformin (dimethylbiguanide); phemformin (phenethylbiguanide); antifolate drugs that inhibit AICAR transformylase; thiazolidinediones; 2-Deoxyglucose (2DG); phenobarbital; A-769662; PT1; salicylate; C24; A-769662; D942; and ZLN024.

7. The method of claim 6, wherein the antifolate drug that inhibits AICAR transformylase is methotrexate or pemetrexed.

8. The method of claim 6, wherein the thiazolidinedione is rosiglitazone, pioglitazone, or troglitazone.

9. A pharmaceutical composition comprising an AMPK activator formulated for ocular administration.

10. The composition of claim 9, formulated for topical ocular administration.

11. The composition of claim 10, which is formulated as eye drops, topical eye cream, or topical eye lotion.

12. The composition of claim 9, which is formulated in single use ampules.

13. The composition of claim 12, wherein the composition lacks a preservative.

14. The composition of claim 10, wherein the AMPK activator formulation comprises microcapsules, microemulsions, or nanoparticles.

15. The composition of claim 9, wherein the AMPK activator is selected from the group consisting of 5-Aminoimidazole-4-carboxamide riboside (AICA riboside or AICAR); AICA ribotide (ZMP); guanidine; galegine; metformin (dimethylbiguanide); phemformin (phenethylbiguanide); antifolate drugs that inhibit AICAR transformylase; thiazolidinediones; 2-Deoxyglucose (2DG); phenobarbital; A-769662; PT1; salicylate; C24; A-769662; D942; and ZLN024.

16. The composition of claim 15, wherein the antifolate drug that inhibits AICAR transformylase is methotrexate or pemetrexed.

17. The composition of claim 15, wherein the thiazolidinedione is rosiglitazone, pioglitazone, or troglitazone.

18. (canceled)

19. A container for drop-wise dispensation of the pharmaceutical composition of claim 9 into the eye of a subject.


SEQUENCE LISTING (3 sequences)

SEQ ID NO: 1 - DNA, 23 bp, Artificial Sequence
  synthetically generated PCR primers
gcttagcacgttaccctggatgg

SEQ ID NO: 2 - DNA, 23 bp, Artificial Sequence
  synthetically generated PCR primers
gttatcagcccaactaattacac

SEQ ID NO: 3 - DNA, 23 bp, Artificial Sequence
  synthetically generated PCR primers
gcattgaaccacagtccttcctc